(19) United States
(12) Patent Application Publication
(10) Pub. No.: US 2010/0065749 A1
(43) Pub. Date: Mar. 18, 2010

(54) RADIOTHERAPY APPARATUS USING TRANSMISSION TYPE DOSIMETER

(76) Inventors: Shinji Nomura, Hiroshima (JP); Tatsufumi Aoi, Hiroshima (JP); Ichiro Yamashita, Hiroshima (JP); Yoshio Sugimoto, Hiroshima (JP); Yuichiro Kamino, Aichi (JP)

Correspondence Address: WENDEROTH, LIND & PONACK, L.L.P., 1030 15th Street, N.W.,, Suite 400 East, Washington, DC, 20005-1503, US

(21) Appl. No.: 12/542,877

(22) Filed: Aug. 18, 2009

(30) Foreign Application Priority Data
Aug. 27, 2008 (JP) 2008-218861

(51) Int. Cl. G01T 1/185; H01J 47/00; A61N 5/00

(52) U.S. Cl. 250/389; 250/492.1; 29/592.1

(57) ABSTRACT

A transmission type dosimeter includes electrodes configured to collect charged particles ionized with radiation, a body, in a cavity of which, the electrodes are arranged, and a lid configured to seal the cavity in the body. The lid includes a fixing frame section fixed on the body, and a transmission section formed with the fixing frame section as a unit body. The transmission section is thinner than the fixing frame section.

### INCORPORATION BY REFERENCE

This application claims a priority on convention based on Japanese Patent Application No. 2008-218861. The disclosure thereof is incorporated herein by reference.

### BACKGROUND OF THE INVENTION

1. Field of the Invention

The present invention relates to a radiotherapy apparatus using a transmission-type dosimeter, and especially relates to a transmission type dosimeter for measuring a dose of radiation.

2. Description of Related Art

A radiotherapy apparatus is known which treats a patient by irradiating a therapeutic radiation to an affected region (tumor). The radiotherapy is desired to accurately irradiate only a predetermined dose of the therapeutic radiation to the affected region and to provide high therapeutic effect. The radiotherapy apparatus includes an irradiation head for emitting the therapeutic radiation, and a transmission type dosimeter for measuring a dose of the therapeutic radiation. The radiotherapy apparatus feedback controls the irradiation head on the basis of the dose measured by the transmission type dosimeter so that only a predetermined dose of the therapeutic radiation is irradiated to the affected region.

FIG. 1 shows a well-known transmission type dosimeter. The dosimeter 100 includes a body 101, an upper side fixing frame 102, an upper side transmission member 103, a lower side fixing frame 104, and a lower side transmission member 105. The transmission-type dosimeter 100 includes the body 101 formed in a cylindrical form to partition a cavity. The body 101 is formed to have a ring shape with a side surface to define the cavity. The upper side transmission member 103 is formed of aluminum in a foil shape. The upper side transmission member 103 is arranged on the body 101 to define an upper surface of the cavity. The upper side fixing frame 102 is formed of aluminum in a plate shape, and has an opening 123 formed in the center. The upper side fixing frame 102 is put on the upper side transmission member 103, and fixed to the body 101 through the upper side transmission member 103. The lower side transmission member 105 is formed of aluminum in a foil shape. The lower side fixing frame 104 is formed of aluminum in a plate shape, and has an opening 124 formed in the center in the same manner as in the upper side fixing frame 102. The lower side transmission member 105 is arranged between the body 101 and the lower side fixing frame 104, and the lower side fixing frame 104 is fixed to the body 101 to define a bottom surface of the cavity.

The body 101 has a flat upper side sealing surface 111 on which the upper side transmission member 103 is put to form a flat upper lid sealing surface 112. An upper side groove 113 is formed in the upper side sealing surface 111. The upper side groove 113 extends to surround the cavity in the body 101 of the transmission type dosimeter 100. An O-ring 114 is arranged in the upper side groove 113. The O-ring 114 is formed of an elastic material. When the upper lid sealing surface 112 adheres tightly to the upper side sealing surface 111, the O-ring 114 elastically deforms to tightly seal the cavity in the body 101 of the transmission type dosimeter 100.

The body 101 further has a flat lower side sealing surface 115, on which the lower side sealing surface 115 is put to form a flat lower lid sealing surface 116. A lower side groove 117 is formed in the body lower side sealing surface 115. The lower side groove 117 extends to surround the cavity in the body 101 of the transmission type dosimeter 100. An O-ring 118 is arranged in the lower side groove 117. The O-ring 118 is formed of an elastic material. When the lower lid sealing surface 116 adheres tightly to the lower side sealing surface 115, the O-ring 118 elastically deforms to tightly seal the cavity in the body 101 of the transmission type dosimeter 100.

The transmission type dosimeter 100 further includes a plurality of electrodes 106 and insulators 107. A plurality of electrodes 106 are formed of an electric conductor. The insulators 107 are formed of an insulating material, and are arranged in the cavity. The insulators 107 support the plurality of electrodes 106 in the cavity of the body 101 so that the plurality of electrodes 106 are not electrically connected with each other. The transmission type dosimeter 100 further includes an electric unit (not shown). The electric unit applies a high voltage to the plurality of electrodes 106 and measures each of currents flowing through the plurality of electrodes 106. A dose of radiation transmitting through the transmission type dosimeter 100 is calculated on the basis of the measured current.

The upper side fixing frame 102 is formed to be totally sufficiently thick so as not to deform over a predetermined deformation amount, and is formed to have the thickness of 3.5 mm. The upper side transmission member 103 is formed to be sufficiently thin so that the transmittance of radiation can be a predetermined value or more, and is formed to have the thickness of 0.5 mm.

As shown in FIG. 2, the transmission type dosimeter 100 includes a plurality of bolts 121. The plurality of bolts 121 are inserted into holes formed in the upper side fixing frame 102, are inserted into holes formed in the upper side transmission member 103, and are tightened to female screws formed on the body 101. Accordingly, the upper side fixing frame 102 and the upper side transmission member 103 are fixed to the body 101. The lower side fixing frame 104 and the lower side transmission member 105 are fixed to the body 101 by using a plurality of bolts in the same manner as the upper side fixing frame 102 and the upper side transmission member 103 are fixed. The transmission type dosimeter 100 further includes a plurality of connectors 122. The plurality of connectors 122 isolate the inside of the transmission type dosimeter 100 from the environment, and are used for not electrically connecting the plurality of electrodes 106 to the body 101, the upper side fixing frame 102, and the lower side fixing frame 104, but electrically connecting the plurality of electrodes 106 to wirings that are electrically connected to the electric unit.

It is desired that such a transmission type dosimeter can measure a dose of radiation stably with respect to a change of the environment.

U.S. Pat. No. 5,079,427 discloses a transmission type dosimeter for retaining own amount influenced by an environmental temperature and a pressure change to be constant by additionally having a bag for exclusive use. U.S. Pat. No. 5,079,427 further discloses a transmission type dosimeter in which a lid of the dosimeter flexibly deforms in response to a change of an environmental pressure.

### SUMMARY OF THE INVENTION

An object of the present invention is to provide a transmission type dosimeter for more stably measuring a dose of radiation.

Another object of the present invention is to provide a transmission type dosimeter for more accurately measuring a dose of radiation.

Further another object of the present invention is to provide a radiotherapy apparatus in which a dose of radiation is more accurately controlled.

Further another object of the present invention is to provide a transmission type dosimeter manufacturing method for manufacturing the transmission type dosimeter for more accurately measuring a dose of radiation.

In an aspect of the present invention, a transmission type dosimeter includes electrodes configured to collect charged particles ionized with radiation, a body, in a cavity of which, the electrodes are arranged, and a lid configured to seal the cavity in the body. The lid includes a fixing frame section fixed on the body, and a transmission section formed with the fixing frame section as a unit body. The transmission section is thinner than the fixing frame section.

In another aspect of the present invention, a transmission type dosimeter system includes a transmission type dosimeter configured to measure a dose of radiation, a sensor configured to measure a parameter of environment of the transmission type dosimeter, and a control unit configured to correct the measured dose based on the measured parameter. The transmission type dosimeter includes electrodes configured to collect charged particles ionized with radiation; a body, in a cavity of which, the electrodes are arranged; and a lid configured to seal the cavity in the body. The lid includes a fixing frame section fixed on the body; and a transmission section formed with the fixing frame section as a unit body. The transmission section is thinner than the fixing frame section.

In still another aspect of the present invention, a radiotherapy apparatus includes a transmission type dosimeter configured to measure a dose of radiation; an irradiation head configured to emit therapeutic radiation which transmits the transmission type dosimeter; and a control unit configured to control the irradiation head to change a dose of the emitted therapeutic radiation based on the measured dose. The transmission type dosimeter includes electrodes configured to collect charged particles ionized with the therapeutic radiation; a body in which the electrodes are arranged; and a lid configured to seal an inside of the body. The lid includes a fixing frame section fixed on the body; and a transmission section formed with the fixing frame section as a unit body to transmit the therapeutic radiation. The transmission section is thinner than the fixing frame section.

In yet still another aspect of the present invention, a method of manufacturing a transmission type dosimeter is achieved by producing a lid of a fixing frame section and a transmission section which is thinner than the fixing frame section; and by fixing the lid to a body to seal an inside of the body in which electrodes arranged to collect charged particles ionized with the therapeutic radiation.

A transmission type dosimeter according to the present invention can more stably measure a dose of radiation. A radiotherapy apparatus according to the present invention can more accurately control a dose of therapeutic radiation.

### BRIEF DESCRIPTION OF THE DRAWINGS

FIG. 1 is a cross sectional view showing a conventional transmission type dosimeter;

FIG. 2 is a plan view showing the conventional transmission type dosimeter;

FIG. 3 is a perspective view showing a radiotherapy apparatus to which a transmission type dosimeter according to the present invention is applied;

FIG. 4 is a cross sectional view showing an irradiation head;

FIG. 5 is a cross sectional view showing the transmission type dosimeter according to the present invention;

FIG. 6 is a plan view showing the transmission type dosimeter according to the present invention;

FIG. 7 is a plan view showing the transmission type dosimeter according to the present invention;

FIG. 8 is a block diagram showing a control unit used in the radiotherapy apparatus according to the present invention;

FIG. 9 is a graph showing a relationship of three factors: an environment pressure, a board thickness of a transmission portion, and a maximum bending amount of the transmission portion;

FIG. 10 is a graph showing variation of measured values in a comparison example of the transmission type dosimeter and variation of measured values according to an embodiment of the transmission type dosimeter; and

FIG. 11 is a graph showing a relationship between the board thickness of the transmission portion and an X-ray transmittance.

### DESCRIPTION OF THE PREFERRED EMBODIMENTS

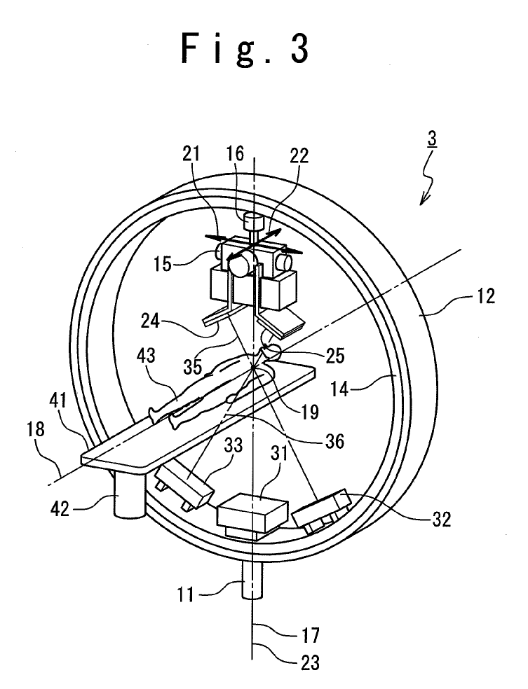

Hereinafter, a transmission type dosimeter according to the present invention will be described with reference to that attached drawings. As shown in FIG. 3, the transmission type dosimeter is applied to a radiotherapy apparatus 3. The radiotherapy apparatus 3 includes a rotation driving unit 11, an O-ring 12, a traveling gantry 14, a swing mechanism 15, and an irradiation head 16. The rotation driving unit 11 supports the O-ring 12 on a base so that the O-ring 12 can rotates around a rotational axis 17, rotates the O-ring 12 around the rotational axis 17 under control of a radiotherapy apparatus control unit (not shown), and outputs a rotation angle of the O-ring 12 with respect to the base. The rotational axis 17 is parallel to a vertical direction. The O-ring 12 is formed in a ring shape including a rotational axis 18 as the center, and supports the traveling gantry 14 so that the traveling gantry 14 can rotate around the rotational axis 18. The rotational axis 18 is orthogonal to the vertical direction, and passes through an isocenter 19 included in the rotational axis 17. The rotational axis 18 is further fixed to the O-ring 12, namely, rotates around the rotational axis 17 with the O-ring 12. The traveling gantry 14 is formed in a ring shape including the rotational axis 18 as the center, and is arranged to form a concentric circle with the circle of the O-ring 12. The radiotherapy apparatus 3 further includes a travel driving unit (not shown). The travel driving unit rotates the traveling gantry 14 around the rotational axis 18 under the control of the radiotherapy apparatus control unit, and outputs a traveling angle of the traveling gantry 14 with respect to the O-ring 12.

The swing mechanism 15 is fixed to the inside of the ring of the traveling gantry 14, and supports the irradiation head 16 on the traveling gantry 14 so that the irradiation head 16 can be arranged to the inside of the traveling gantry 14. The swing mechanism 15 has a pan axis 21 and a tilt axis 22. The pan axis 21 is fixed to the traveling gantry 14, and is parallel to the rotational axis 18 without intersecting with the rotational axis 18. The tilt axis 22 is orthogonal to the pan axis 21. The swing mechanism 15 swings the irradiation head 16 around the pan axis 21 and the irradiation head 16 around the tilt axis 22 under the control of the radiotherapy apparatus control unit.

The irradiation head 16 irradiates a therapeutic radiation 23 under the control of the radiotherapy apparatus control unit. The therapeutic radiation 23 is irradiated almost along a straight line passing through an intersection point where the pan axis 21 intersects with the tilt axis 22. The therapeutic radiation 23 is formed to have an even intensity distribution. The therapeutic radiation 23 is further partially shielded to control a shape of irradiation field when the therapeutic radiation 23 is irradiated to a patient. When the irradiation head 16 is supported by the traveling gantry 14 in this manner and the irradiation head 16 is once adjusted to face the isocenter 19 by the swing mechanism 15, the therapeutic radiation 23 constantly passes through almost the isocenter 19 even if the O-ring 12 is rotated by the rotation driving unit 11 or the traveling gantry 14 is rotated by the travel driving unit. That is, the traveling and rotation can realize the irradiation of the therapeutic radiation 23 from an arbitrary direction to the isocenter 19.

The radiotherapy apparatus 3 further includes a plurality of imager systems. That is, the radiotherapy apparatus 3 includes diagnostic X-ray sources 24 and 25 and sensor arrays 32 and 33. The diagnostic X-ray source 24 is supported on the traveling gantry 14. The diagnostic X-ray source 24 is arranged on the inside of the ring of the traveling gantry 14, so that an angle between a line segment connecting the isocenter 19 to the diagnostic X-ray source 24 and a line segment connecting the isocenter 19 to the irradiation head 16 is an acute angle. The diagnosis X-ray source 24 emits a diagnosis X-ray 35 to the isocenter 19 under the control of the radiotherapy apparatus control unit. The diagnosis X-ray 35 is emitted from one point included in the diagnosis X-ray source 24, and has a corn beam in a conical shape including the one point. The diagnostic X-ray source 25 is supported on the traveling gantry 14. The diagnostic X-ray source 25 is arranged at the inside of the ring of the traveling gantry 14, so that an angle between a line segment connecting the isocenter 19 to the diagnostic X-ray source 25 and a line segment connecting the isocenter 19 to the irradiation head 16 is an acute angle. The diagnosis X-ray source 25 emits a diagnosis X-ray 36 to the isocenter 19 under the control of the radiotherapy apparatus control unit. The diagnosis X-ray 36 is emitted from one point of the diagnosis X-ray source 25, and has a corn beam in a conical shape including the one point.

The sensor array 32 is supported on the traveling gantry 14. The sensor array 32 receives the diagnosis X-ray 35 that is emitted from the diagnosis X-ray source 24 and transmits through an object surrounding the isocenter 19, and generates a transmission image of the object. The sensor array 33 is supported on the traveling gantry 14. The sensor array 33 receives the diagnosis X-ray 36 that is emitted from the diagnosis X-ray source 25 and transmits through an object surrounding the isocenter 19, and generates a transmission image of the object. An FPD (Flat Panel Detector) and an X-ray II (image Intensifier) are exemplified as the sensor arrays 32 and 33. According to such an imager system, a transmission image around the isocenter 19 can be generated on the basis of image signals obtained by the sensor arrays 32 and 33.

The radiotherapy apparatus 3 further includes a sensor array 31. The sensor array 31 is arranged on the inside of the ring of the traveling gantry 14 so that a line segment connecting the sensor array 31 to the irradiation head 16 passes through the isocenter 19. The sensor array 31 receives the therapeutic radiation 23 that is emitted from the irradiation head 16 and transmits through an object surrounding the isocenter 19, and generates a transmission image of the object. An FPD (Flat Panel Detector) and an X-ray II (image Intensifier) are exemplified as the sensor array 31.

The radiotherapy apparatus 3 further includes a couch 41 and a couch driving unit 42. The couch 41 is used by a patient 43 so as to lie on it, who will be treated by the radiotherapy apparatus 3. The couch 41 includes a fixture (not shown). The fixture fixes the patient on the couch 41 so that the patient cannot move on it. The couch driving unit 42 supports the couch 41 on the base, and moves the couch 41 under the control of the radiotherapy apparatus control unit.

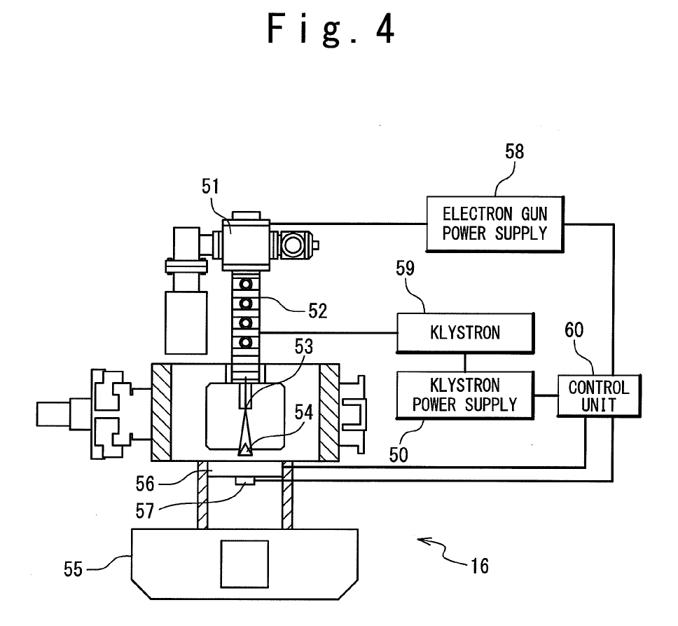

FIG. 4 shows the irradiation head 16. The irradiation head 16 includes an electron gun 51, an acceleration tube 52, an X-ray target 53, a flattening filter 54, and a multi-leaf collimator 55. The electron gun 51 emits electrons. The acceleration tube 52 accelerates the electrons emitted from the electron gun 51 to generate an electron beam, and irradiates the electron beam to the X-ray target 53. The X-ray target 53 is formed of a material with a large atomic number. As such a material, tungsten, tungsten alloy, gold, tantalum and the like are exemplified. The X-ray target 53 generates a radiation (X-ray) because of the bremsstrahlung of the electron beam generated by the acceleration tube 52. The radiation is irradiated almost along a straight line passing through a virtual radiation point source that is a point of the X-ray target 53. The flattening filter 54 is formed of aluminum and the like in a plate shape on which approximately-conical projections are formed. The flattening filter 54 is arranged so that the projections face a side of the X-ray target 53. The flattening filter 54 is formed so that after a radiation irradiated from the X-ray target 53 has passed through the flattening filter 54, a dose of radiation in a predetermined region on an isocenter plane orthogonal to the irradiation direction has an almost uniform distribution. The multi-leaf collimator 55 partially shields the radiation transmitting through the flattening filter 54 to control a shape of irradiation field when the therapeutic radiation 23 is irradiated to a patient under the control of the radiotherapy apparatus control unit.

The radiotherapy apparatus 3 further includes a transmission type dosimeter 56, a sensor 57, an electron gun power supply 58, a klystron power supply 50, a klystron 59, and a control unit 60. The transmission type dosimeter 56, the sensor 57, the electron gun power supply 58, the klystron power supply 50, and the klystron 59 are connected to the control unit 60 to e communicable with the control unit 60. The transmission type dosimeter 56 is arranged to transmit the radiation transmitting through the flattening filter 54. The transmission type dosimeter 56 measures a dose of the transmitting radiation and outputs the measured dose to the control unit 60. The sensor 57 is arranged in the vicinity of the transmission type dosimeter 56 so as not to be irradiated by the radiation. The sensor 57 measures an atmospheric pressure of the environment where the transmission type dosimeter 56 is arranged, and outputs the measured atmospheric pressure to the control unit 60. The electron gun power supply 58 supplies a predetermined power to the electron gun 51 under control of the control unit 60. The klystron power supply 50 supplies power to the klystron 59 under the control of the control unit 60. The klystron 59 is connected to the acceleration tube 52 via a waveguide. Under the control of the control unit 60, the klystron 59 generates a predetermined power by using the power supplied from the klystron power supply 50 and supplies the generated power to the acceleration tube 52 via the waveguide. Meanwhile, in place of the klystron 59 another high frequency source may be used. As the high frequency source, the magnetron and the multielectrode tube are exemplified.

The control unit 60 is a computer, and includes a CPU, a storage unit, an input unit, an output unit, and an interface (they are not shown). The CPU executes computer programs loaded in the control unit 60 to control the storage unit, the input unit, the output unit, and the interface. The storage unit stores the computer programs, and temporarily stores data generated by the CPU. The input unit generates data through an operation by user and outputs the data to the CPU. A keyboard is exemplified as the input unit. The output unit outputs the data generated by the CPU to the user so that the data can be visible. A display is exemplified as the output unit. The interface outputs data generated by an external unit connected to the control unit 60 to the CPU, and outputs the data generated by the CPU to the external unit. The external unit includes the transmission type dosimeter 56, the sensor 57, the electron gun power supply 58, the klystron power supply 50, and the klystron 59.

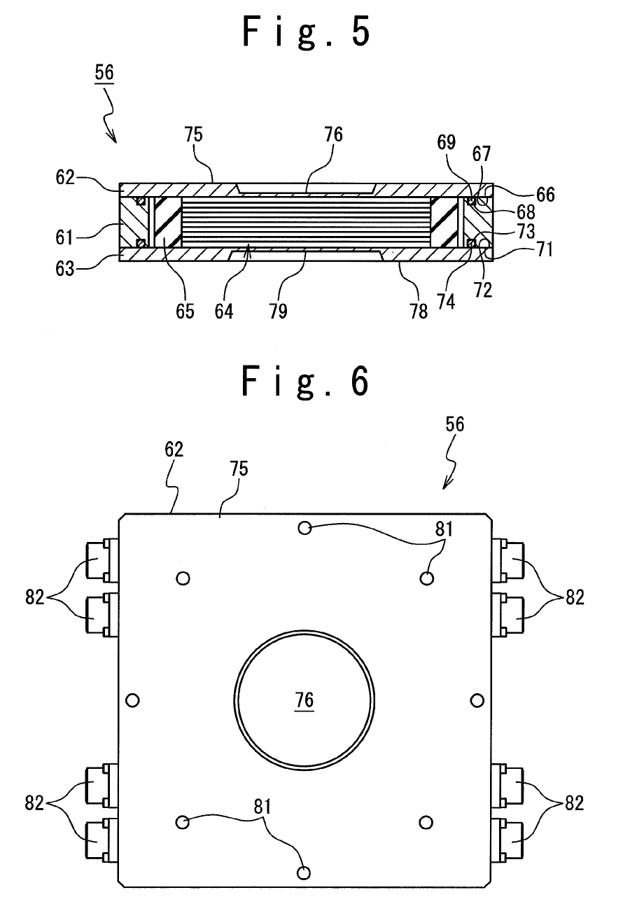

FIG. 5 shows the transmission type dosimeter 56. The transmission type dosimeter 56 includes a body 61, an upper lid 62, and a lower lid 63. The body 61 has a cylindrical shape to define a side wall of a cavity. The upper lid 62 is formed of aluminum in a plate shape. The upper lid 62 forms an upper surface wall of the cavity. The lower lid 63 is formed of aluminum in a plate shape. The lower lid 63 forms a lower surface wall of the cavity. The transmission type dosimeter 56 is arranged in the irradiation head 16 so that the upper lid 62 can be arranged on a side of the flattening filter 54.

The body 61 has a flat upper side sealing surface 66. The upper lid 62 has a flat upper lid sealing surface 67. An upper side groove 68 is formed in the upper side sealing surface 66, and extends to surround the cavity of the transmission type dosimeter 56. An O-ring 69 is arranged in the upper side groove 68. The O-ring 69 is formed of an elastic material. When the upper lid sealing surface 67 adheres tightly to the upper side sealing surface 66, the O-ring 69 elastically deforms to tightly seal the internal portion of the container of the transmission type dosimeter 56. The body 61 further has a flat lower side sealing surface 71. The lower lid 63 has a flat lower lid sealing surface 72. A lower side groove 73 is formed in the lower side sealing surface 71. The lower side groove 73 extends to surround the cavity of the transmission type dosimeter 56. An O-ring 74 is arranged in the lower side groove 73. The O-ring 74 is formed of the elastic material. When the lower lid sealing surface 72 adheres tightly to the lower side sealing surface 71, the O-ring 74 elastically deforms to tightly seal the internal portion of the container of the transmission type dosimeter 56.

The transmission type dosimeter 56 further includes a plurality of electrodes 64 and insulator sections 65. The plurality of electrodes 64 are formed of electric conductor, and are arranged in the cavity. The insulator section 65 is formed of an insulating material, and is arranged in the cavity. The insulator section 65 supports the plurality of electrodes 64 with respect to the body 61 so that the plurality of electrodes 64 are not electrically connected with each other. The plurality of electrodes 64 include positive electrodes and negative electrodes. The positive electrodes are arranged on positions separated from each other, and also, the negative electrodes are arranged on positions separate from each other. The transmission type dosimeter 56 further includes an electronic unit (not shown). The electronic unit applies a high voltage between the positive electrodes and the negative electrodes, measures current flowing between each of the positive electrodes and one of the negative electrodes, and outputs data of the measured current to the control unit 60. In this case, the transmission type dosimeter 56 can detect positions of charged particles ionized due to a radiation. A method of detecting position of radiation by using the plurality of electrodes 64 is well known as the PSD (Position Sensitive Detector). The transmission type dosimeter of the present invention has a same or similar structure as or to those disclosed in U.S. Pat. Nos. 4,431,921, 4,827,135, and 4,965,861.

It should be noted that a transmission type dosimeter may be used that can detect only a dose of the radiation without detecting a position of transmitting radiation. In this case, the electronic unit applies high voltages between the positive electrodes and the negative electrodes, measures currents flowing between the positive electrodes and the negative electrodes, and outputs data of the measured currents to the control unit 60.

As shown in FIG. 6, the upper lid 62 is formed of a fixing frame portion 75 and a transmission portion 76. The fixing frame portion 75 is arranged to surround the transmission portion 76. In the fixing frame portion 75, the upper lid 62 is formed to be totally sufficiently thick, i.e. to have the thickness of 5 mm, so that it does not deform over a predetermined deformation amount. The transmission portion 76 is formed to have a circular shape of the diameter of 70 mm. The transmission portion 76 is formed to be thinner than the fixing frame portion 75, i.e. to have the thickness of 1 mm. More specifically, the transmission portion 76 is formed to be sufficiently thin so that the transmittance of radiation can be a predetermined amount or more, and the shape of the cavity of the transmission type dosimeter 56 does not deform over the predetermined deformation amount within a predetermined range of atmospheric pressure. The transmission type dosimeter 56 further includes a plurality of bolts 81. The plurality of bolts 81 are inserted into holes formed in the fixing frame portion 75 of the upper lid 62, are tightened to female screws formed in the body 61. Accordingly, the upper lid 62 is directly fixed or coupled to the body 61.

The transmission type dosimeter 56 further includes a plurality of connectors 82. The connectors 82 isolate the inside of the transmission type dosimeter 56 from the environment, and are used not to electrically connect the plurality of electrodes 64 to the body 61, the upper lid 62, and the lower lid 63, but to electrically connect the plurality of electrodes 64 to wirings that are electrically connected to the electronic unit.

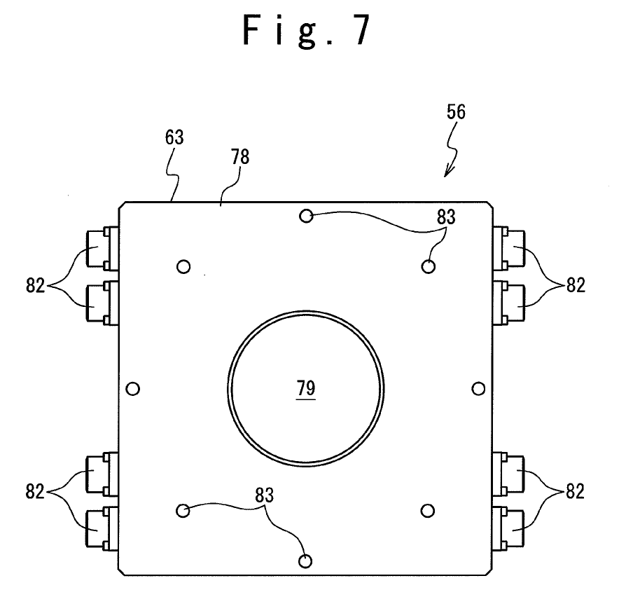

As shown in FIG. 7, the lower lid 63 is formed of a fixing frame portion 78 and a transmission portion 79. The fixing frame portion 78 is arranged to surround the transmission portion 79. In the fixing frame portion 78, the lower lid 63 is formed to be totally sufficiently thick, i.e. to have the thickness of 5 mm so that it does not deform over a predetermined deformation amount. The transmission portion 79 is formed to be a circular shape of the diameter of 80 mm. The transmission portion 79 is formed to be thinner than the fixing frame portion 78, i.e. to have the thickness of 1 mm. More specifically, the transmission portion 79 is formed to be sufficiently thin so that the transmittance of radiation can be a predetermined amount or more, and the shape of the cavity of the transmission type dosimeter 56 does not deform over the predetermined deformation amount within a predetermined range of atmospheric pressure. The transmission type dosimeter 56 further includes a plurality of bolts 83. The plurality of bolts 83 are inserted into holes formed in the fixing frame portion 78 of the lower lid 63, and are tightened to the female screws formed on the body 61. Accordingly, the lower lid 63 is directly fixed or coupled to the body 61.

The transmission portion 79 is formed so that a solid angle of the transmission portion 79 from a virtual radiation point source of the X-ray target 53 is equal to a solid angle of the transmission portion 76 from the virtual radiation point source, and preferably is formed to be slightly wider. That is, the lower lid 63 is formed so that radiation emitted from the virtual radiation point source and transmitting through the transmission portion 76 can transmit through the transmission portion 79, and the transmission portion 79 is formed to be equal to or wider than the transmission portion 76. Here, the size of the transmission type dosimeter 56 described in the description is only an example, the dosimeter is appropriately designed to satisfy the conditions described in the description.

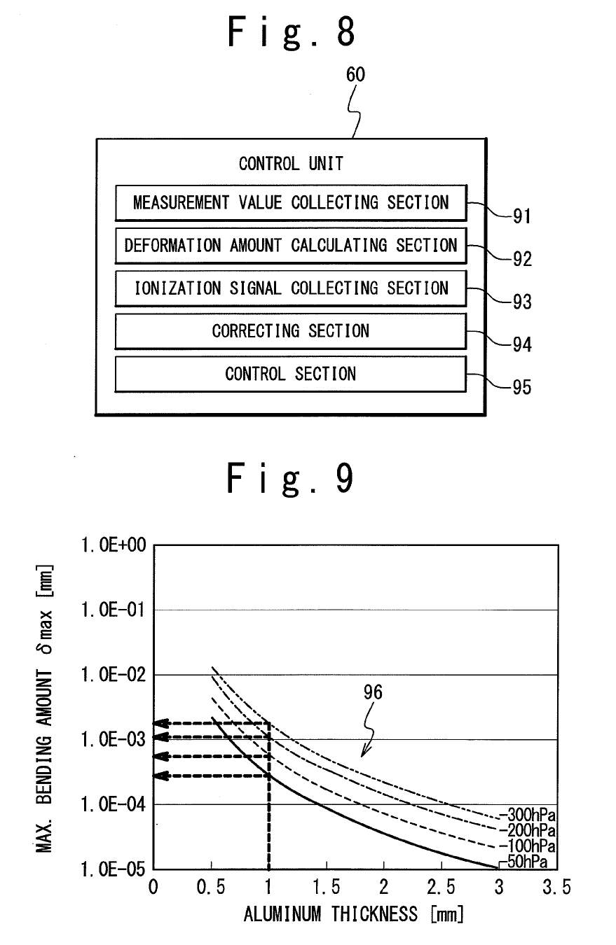

As shown in FIG. 8, a computer program loaded in the control unit 60 includes a measurement value collecting section 91, a deformation amount calculating section 92, an ionization signal collecting section 93, a correcting section 94, and a control section 95. The measurement value collecting section 91 measures an atmospheric pressure of environment where the transmission type dosimeter 56 is arranged, by using the sensor 57, and collects the measured atmospheric pressure from the sensor 57. The deformation amount calculating section 92 calculates a deformation amount of the cavity of the transmission type dosimeter 56 on the basis of the atmospheric pressure collected by the measurement value collecting section 91. The deformation amount represents a deformation amount of the upper lid 62 of the transmission type dosimeter 56 and a deformation amount of the lower lid 63. The ionization signal collecting section 93 measures a dose of radiation transmitting through the transmission type dosimeter 56 by using the transmission type dosimeter 56, and collects the measured dose from the transmission type dosimeter 56. That is, the ionization signal collecting section 93 measures each of currents flowing through the positive electrodes and negative electrodes of the electrodes 64, and collects the measured currents from the transmission type dosimeter 56. The ionization signal collecting section 93 calculates a total of doses of radiation transmitting through the transmission type dosimeter 56 on the basis of the measured currents, and calculates a distribution of doses of radiation transmitting through the transmission type dosimeter 56.

The correcting section 94 corrects the total of doses calculated by the ionization signal collecting section 93 on the basis of the deformation amount calculated by the deformation amount calculating section 92 to obtain a corrected dose. The control section 95 controls the electron gun power supply 58, the klystron power source 50, and the klystron 59 in a feedback manner on the basis of the corrected dose calculated by the correcting section 94. For example, the control section 95 controls the electron gun power supply 58, the klystron power source 50, and the klystron 59 to reduce variations of the corrected dose. For example, the control section 95 updates power supplied to the electron gun 51 by controlling the electron gun power supply 58 and updates power supplied to the acceleration tube 52 by controlling the klystron power source 50, and the klystron 59 to reduce variations of the corrected dose.

A transmission type dosimeter manufacturing method according to an embodiment of the present invention includes an operation of manufacturing the upper lid 62 and the lower lid 63, and an operation of fixing the upper lid 62 and the lower lid 63 to the body 61. Specifically, the upper lid 62 is manufactured in a carving-out process as a unitary body of the fixing frame portion 75 and the transmission portion 76 without being disassembled. In the same manner, the lower lid 63 is manufactured in the carving-out process as a unitary body of the fixing frame portion 78 and the transmission portion 79 without being disassembled. It should be noted that the upper lid 62 and the lower lid 63 may be manufactured through another machining process other than the carving-out process. Machining processes such as casting, forging, and welding of a plurality of parts are exemplified.

The upper lid 62 is fixed to the body 61 by inserting the plurality of bolts 81 into the plurality of holes formed in the fixing frame portion 75, respectively, and by tightening the plurality of bolts 81 to the female screws formed in the body 61. In the same manner as in the upper lid 62, the lower lid 63 is fixed to the body 61 by inserting the plurality of bolts 83 into the plurality of holes formed in the fixing frame portion 78, respectively, and by tightening the plurality of bolts 83 to the female screws formed on the body 61.

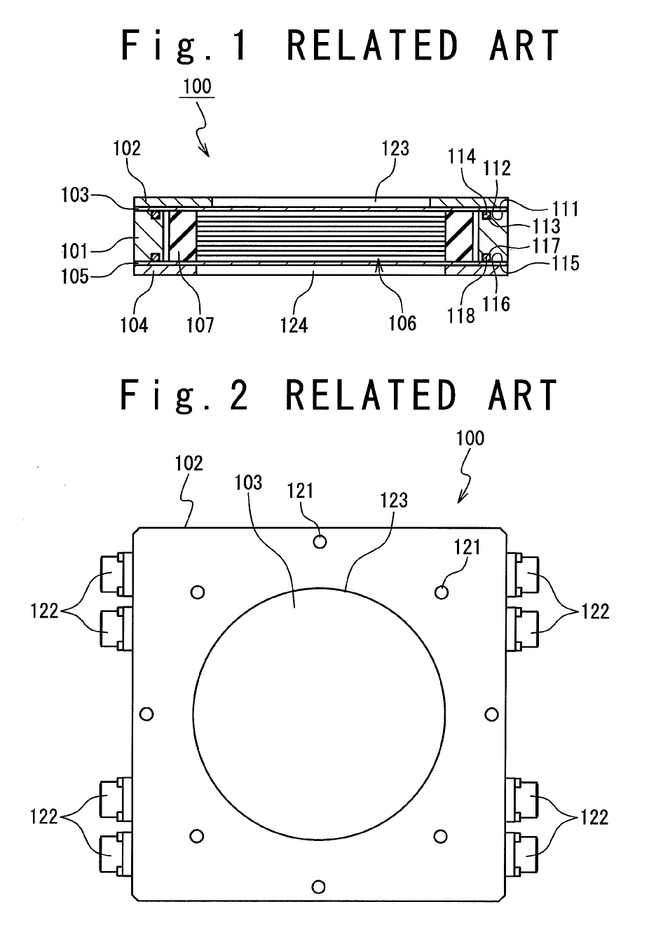

When manufactured in this manner, the upper lid 62 and the lower lid 63 attain improved rigidity compared to a lid that can be disassembled. Accordingly, the upper lid 62 and the lower lid 63 are prevented from deforming due to the atmospheric pressure of environment where the transmission type dosimeter 56 is arranged, and the transmission type dosimeter 56 prevents a density of gas filled in the cavity from changing. For this reason, the transmission type dosimeter 56 can measure the dose of transmitting radiation more stably. Compared to the transmission type dosimeter 100 shown in FIGS. 1 and 2, the transmission type dosimeter 56 can further reduce time and a work amount, and thus can be manufactured more easily.

FIG. 9 shows a relationship of an atmospheric pressure of environment where the transmission type dosimeter 56 is arranged, a plate thickness of the transmission portion 76; and a maximum bending amount of the transmission portion 76. In FIG. 9, the atmospheric pressure is represented on the basis of an atmospheric pressure difference from 1 atmosphere (1013 hPa). When the upper lid 62 is modeled by a beam model, the maximum bending amount shows a degree of bending of the beam model. When an absolute value of the atmospheric pressure difference is same, the maximum bending amount generally takes a same value in both cases where the atmospheric pressure rises and drops. The relationship 96 shows that as the absolute value of the atmospheric pressure difference becomes larger, the maximum bending amount of the transmission portion 76 becomes larger. The relationship 96 further shows that as the plate thickness of the transmission portion 76 becomes thinner, the maximum bending amount becomes larger. That is, the relationship 96 shows that the transmission portion 76 of the transmission type dosimeter 56 is hard to be deformed based on the change of atmospheric pressure compared to the upper side transmission member 103 or the lower side transmission member 105 of the transmission type dosimeter 100 shown in FIGS. 1 and 2. The relationship 96 further shows that the maximum bending amount is smaller than a predetermined value (approx. 2.0×10⁻³ mm) within a range of atmospheric pressure (7×10⁴ Pa to 11×10⁴ Pa) defined by the IEC standards, when the plate thickness of the transmission portion 76 is approximately 1 mm or more.

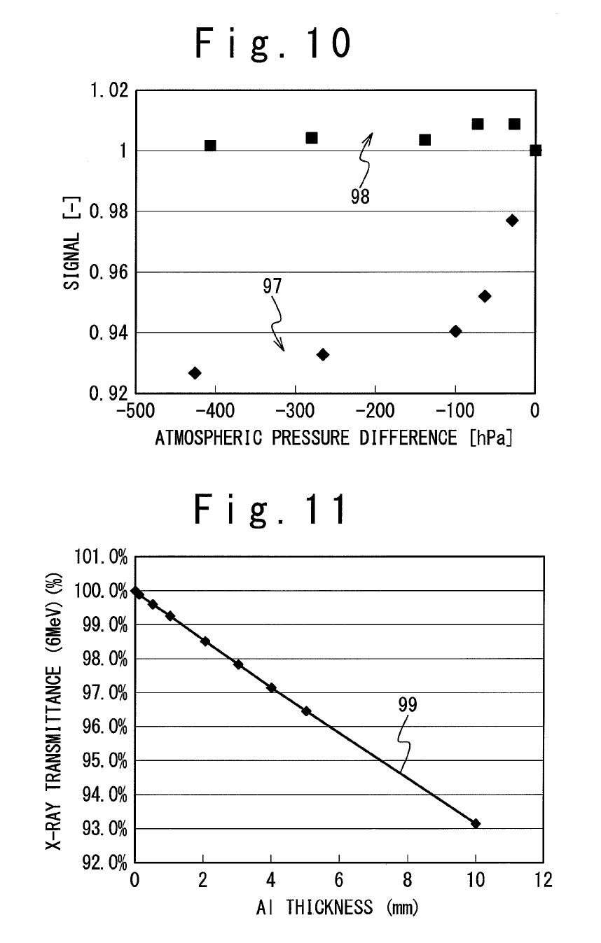

FIG. 10 shows variations of measured values of dose in a comparison example due to variations of an atmospheric pressure of environment where a transmission type dosimeter of the comparison example is arranged. The comparison example is same as the transmission type dosimeter 100 shown in FIGS. 1 and 2. The atmospheric pressure is represented on the basis of an atmospheric pressure difference from 1 atmosphere (1013 hPa). The variations 97 show that measured values of the transmission type dosimeter 100 relatively largely change due to the change of atmospheric pressure. In addition, the variations 97 and the relationship 96 of FIG. 9 show that values of dose measured by the transmission type dosimeter 100 vary when the upper side transmission member 103 or the lower side transmission member 105 of the transmission type dosimeter 100 bends. FIG. 10 further shows variations of the values of dose measured by the transmission type dosimeter 56 with respect to variations of the atmospheric pressure of environment where the transmission type dosimeter 56 is arranged. The variations 98 show that variations of the measured values of dose of the transmission type dosimeter 56 with respect to the variations of atmospheric pressure is smaller than those of the comparison example of the transmission type dosimeter. The variations 98 further show that the variations of the measured values of the transmission type dosimeter 56 (a standard deviation) are smaller than a predetermined value (2% recommended by the JIS standards) within the range of atmospheric pressure defined by the IEC standards. The variations 98 further show that within the range of atmospheric pressure defined by the IEC standards, the transmission type dosimeter 56 can sufficiently-accurately measure a dose of radiation within the range of atmospheric pressure.

FIG. 11 shows a relationship between the plate thickness of the transmission portion 76 and the X-ray transmittance in the transmission type dosimeter 56. The relation 99 shows that as the plate thickness of the transmission portion 76 becomes thinner, the X-ray transmittance of the transmission type dosimeter 56 becomes larger. The relationship 99 further shows that variations of the X-ray transmittance in the transmission type dosimeter 56 is within the predetermined range (±2%) when the plate thickness of the transmission portion 76 varies when the plate thickness of the transmission portion 76 is thinner than approximately 1 mm. That is, the relationship 99 shows that the transmission type dosimeter 56 can sufficiently accurately measure a dose of radiation within the range when the plate thickness of the transmission portion 76 is thinner than approximately 1 mm.

In the radiotherapy using the radiotherapy apparatus 3, a user firstly produces a therapeutic plan. The therapeutic plan shows irradiation angles at which the therapeutic radiation 23 is irradiated to an affected region of the patient 43, and a dose and a property of the therapeutic radiations 23 irradiated at the respective irradiation angles. The user fixes the patient 43 to the couch 41 of the radiotherapy apparatus 3. The radiotherapy apparatus control unit of the radiotherapy apparatus 3 aligns positions of the irradiation head 16 and the patient 43 by using the rotation driving unit 11, the travel driving unit and the couch driving unit 42, to irradiate the therapeutic radiation 23 to the patient 43 at the irradiation angles shown in the therapeutic plan.

Subsequently, the radiotherapy apparatus control unit repeatedly carries out a tracking operation and an irradiation operation. In the tracking operation, the radiotherapy apparatus control unit calculates a position of the affected region on the basis of images taken by the imager system of the radiotherapy apparatus 3. The radiotherapy apparatus control unit drives the irradiation head 16 by using the swing mechanism 15 so that the therapeutic radiation 23 can transmit through the affected region. In the irradiation operation, the radiotherapy apparatus control unit irradiates the therapeutic radiation 23 to the affected region by using the irradiation head 16 immediately after the irradiation head 16 is moved in the tracking operation.

The control unit 60 collects a does of radiation transmitting through the transmission type dosimeter 56 from the transmission type dosimeter 56 during the repeated execution of the tracking operation and the irradiation operation. The control unit 60 further collects an atmospheric pressure from the sensor 57, and calculates a deformation amount of the cavity of the transmission type dosimeter 56 on the basis of the atmospheric pressure. The control unit 60 corrects the collected dose on the basis of the calculated deformation amount, and controls the electron gun power supply 58, the klystron power supply 50 and the klystron 59 in a feedback manner on the basis of the corrected dose. Specifically, the control unit 60 updates power supplied to the electron gun 51 by controlling the electron gun power supply 58 to reduce variations of the corrected dose, and updates power supplied to the acceleration tube 52 by controlling the klystron power source 50, and the klystron 59.

According to such operations, the irradiation head 16 can reduce variations of the dose of the therapeutic radiation 23, and the radiation therapeutic apparatus 3 can more accurately irradiate only a predetermined dose of the therapeutic radiation 23 to the affected region of the patient 43.

It should be noted that another sensor may be used which measures another measurement value other than the atmospheric pressure in place of the sensor 57. Or, a plurality of sensors may be used to respectively measure a plurality of measurement values. As the measurement value, temperature of environment where the transmission type dosimeter 56 is arranged, temperatures of the upper lid 62 and the lower lid 63 in the transmission type dosimeter 56, or deformation amounts themselves of the upper lid 62 and the lower lid 63 in the transmission type dosimeter 56 are exemplified. In this case, the control unit 60 controls the electron gun power supply 58, the klystron power supply 50 and the klystron 59 in a feedback manner on the basis of the measured value, in the same manner as in the above embodiments. Such a radiotherapy apparatus can more accurately irradiate only a predetermined dose of the therapeutic radiation to the affected region of a patient in the same manner as in the above embodiments.

The transmission type dosimeter 56 can be applied to another unit that is different from the radiotherapy apparatus 3. For example, it can be singly used. In such a case, the control unit 60 outputs a dose corrected on the basis of the measured value to the user via an output unit so that the dose is visible.

What is claimed is:

1. A transmission type dosimeter comprising:
electrodes configured to collect charged particles ionized with radiation;
a body, in a cavity of which, said electrodes are arranged; and
a lid configured to seal the cavity in said body,
wherein said lid comprises:
a fixing frame section fixed on said body; and
a transmission section unitarily formed with said fixing frame section, and
said transmission section is thinner than said fixing frame section.

2. The transmission type dosimeter according to claim 1, further comprising:
an opposite-side lid configured to seal the cavity in said body,
wherein said opposite-side lid comprises:
an opposite-side fixing frame section fixed on said body; and
an opposite-side transmission section unitarily formed with said opposite-side fixing frame section,
wherein said opposite-side lid is on an opposite side to said lid with respect to said electrodes, and
said opposite-side transmission section is thinner than said opposite-side fixing frame section.

3. The transmission type dosimeter according to claim 2, wherein said opposite-side transmission section is equal to or wider than said transmission section.

4. A transmission type dosimeter system comprising:
a transmission type dosimeter configured to measure a dose of radiation;
a sensor configured to measure a parameter of environment of said transmission type dosimeter; and
a control unit configured to correct the measured dose based on said measured parameter,
wherein said transmission type dosimeter comprises:
electrodes configured to collect charged particles ionized with radiation;
a body, in a cavity of which, said electrodes are arranged; and
a lid configured to seal the cavity in said body;
wherein said lid comprises:
a fixing frame section fixed on said body; and
a transmission section unitarily formed with said fixing frame section, and
said transmission section is thinner than said fixing frame section.

5. The transmission type dosimeter system according to claim 4, wherein said transmission type dosimeter further comprises:
an opposite-side lid configured to seal the cavity in said body,
wherein said opposite-side lid comprises:
an opposite-side fixing frame section fixed on said body; and
an opposite-side transmission section unitarily formed with said opposite-side fixing frame section,
wherein said opposite-side lid is on an opposite side to said lid with respect to said electrodes, and
said opposite-side transmission section is thinner than said opposite-side fixing frame section.

6. The transmission type dosimeter system according to claim 5, wherein said opposite-side transmission section is equal to or wider than said transmission section.

7. The transmission-type dosimeter system according to claim 4, wherein said measured parameter is an atmospheric pressure of the environment where said transmission type dosimeter is arranged.

8. The transmission type dosimeter system according to claim 4, wherein said measured parameter is a temperature of the environment of said transmission type dosimeter.

9. The transmission type dosimeter system according to claim 4, wherein said measured parameter indicates a deformation of said lid.

10. The transmission type dosimeter system according to claim 4, wherein further comprising:
counter electrodes, each of which is provided for one of said electrodes,
wherein a bias is applied between the counter electrode and one of said electrode.

11. A radiotherapy apparatus comprising:
a transmission type dosimeter configured to measure a dose of radiation;
an irradiation head configured to emit therapeutic radiation which transmits said transmission type dosimeter; and
a control unit configured to control said irradiation head to change a dose of the emitted therapeutic radiation based on the measured dose,
wherein said transmission type dosimeter comprises:
electrodes configured to collect charged particles ionized with said therapeutic radiation;
a body, in a cavity of which, said electrodes are arranged; and
a lid configured to seal the cavity in said body,
wherein said lid comprises:
a fixing frame section fixed on said body; and
a transmission section unitarily formed with said fixing frame section to transmit said therapeutic radiation, and
said transmission section is thinner than said fixing frame section.

12. The radiotherapy apparatus according to claim 11, wherein said transmission type dosimeter further comprises:
an opposite-side lid configured to seal the cavity in said body,
wherein said opposite-side lid comprises:
an opposite-side fixing frame section fixed on said body; and
an opposite-side transmission section unitarily formed with said opposite-side fixing frame section,
wherein said opposite-side lid is on an opposite side to said lid with respect to said electrodes, and
said opposite-side transmission section is thinner than said opposite-side fixing frame section.

13. The radiotherapy apparatus according to claim 12, wherein said opposite-side transmission section is equal to or wider than said transmission section.

14. The radiotherapy apparatus according to claim 11, further comprising:
a sensor configured to measure data of environment of said transmission type dosimeter, and
wherein said control unit corrects said measured dose, which is calculated based on electric currents which flows through said electrodes, based on said measured data, and controls said irradiation head to change the dose of the emitted therapeutic radiation based on the corrected dose.

15. A method of manufacturing a transmission type dosimeter comprising:
producing a lid of a fixing frame section and a transmission section which is thinner than said fixing frame section; and
fixing said lid to a body to seal an inside of the body in which electrodes arranged to collect charged particles ionized with the therapeutic radiation.
